(19) United States
(12) Patent Application Publication
(10) Pub. No.: US 2025/0157571 A1
(43) Pub. Date: May 15, 2025

(54) Methods, Apparatus, and Systems for Determining the Functional Microbial Niche and Identifying Interventions in Complex Hetergeneous Communities

(71) Applicant: The Regents of the University of California, Oakland, CA (US)

(72) Inventors: Karsten Zengler, La Jolla, CA (US); Mahmoud Al-Bassam, La Jolla, CA (US); Oriane Moyne, San Diego, CA (US)

(21) Appl. No.: 18/838,847

(22) PCT Filed: Jan. 30, 2023
(86) PCT No.: PCT/US2023/061546

§ 371 Date: Aug. 15, 2024

Related U.S. Application Data
(60) Provisional application No. 63/310,476, filed on Feb. 15, 2022.

(51) Int. Cl. G16B 20/00; C12N 1/20; G16B 5/00

(52) U.S. Cl. CPC G16B 20/00; C12N 1/20; G16B 5/00

(57) ABSTRACT

Metagenomics, metatranscriptomics and/or metatranslatomics measurement of the translational efficiency (TE) on genes and metabolic pathways using genome annotation, transcription and translation information of one or more identified member of a microbial consortia for the design and intervention of the microbial consortia. Also provided is the method for predicting the effect of perturbations on one or more members of a microbial consortia and a method for selecting one or more members of a microbial consortia for a particular task.

### CROSS-REFERENCE TO RELATED APPLICATION

This application claims the benefit of U.S. Provisional Application No. 63/310,476, filed on Feb. 15, 2022, the entire content of which is incorporated herein by reference.

### BACKGROUND

Microbiome science has contributed greatly to the understanding of microbial life and provided insights on the essential roles of microbial communities on the planet, from global elements cycling to human health. However, there is still lack of knowledge on how these communities are assembled, maintained, and function as a system. Most importantly, microbe-microbe interactions and how microbes and communities react to perturbations are poorly understood. As a consequence, microbiome science today is mostly descriptive and correlation-based, rather than predictive and based on mechanistic understanding. In order to achieve predictive microbiome science, there is a need to comprehensively elucidate the metabolic role of each microbe, and its interactions with others. Such knowledge would allow to change a microbe's trajectory within a community, for example by selectively promoting or limiting its growth.

Ribo-Seq, a promising technology to study translational regulation, was first applied to yeast in 2009 and then extended to animal stem cells and recently to a handful of model bacteria. The Zengler lab was among the first labs to streamline the protocol and to extend the technology to strictly anaerobic bacteria, like the ones present in the human gut. Recently, Fremin et al. publish a paper that describes also metaRibo-Seq measures translation in microbiomes (Fremin et al., Nature Communications 11, 3268 (2020). However, Fremin's measures using a protocol that does not integrate RNA-Seq with Ribo-Seq to identify TE, determine guilds and microbial niches.

### SUMMARY

Microbes tightly control their genotype-to-phenotype relationship by controlling resource allocation through the complex regulation of their transcription and translation. Determining how a microbe allocates its cellular resources to achieve the optimal balance between mRNA levels and protein numbers reveals its niche. Since translation is the most expensive process in a cell, bacteria use translational regulation to prioritize functions essential for their niche adaptation. Translation efficiency (TE), calculated by analyzing the number of ribosomes on a given transcript, can be used as a direct readout of functional prioritization in pure cultures. TE can be measured using ribosome sequencing (Ribo-Seq), as the ratio between translated mRNA over total mRNA (RNA-Seq). Ribo-seq is based on blocking the ribosomes on the mRNA during the elongation step of mRNA translation to protein, and then sequencing all (and only) the mRNA fragments that are covered (thus actively translated) by the ribosomes. In comparison with proteomics, which involves measuring all the proteins that are present in a sample independently of when these proteins were produced, Ribo-Seq (translatomics) provides a snapshot of active protein translation at a given instant, thus giving a time-resolved view of bacterial resource allocation.

The present disclosure provides a method for a design and intervention of microbial consortia. In certain embodiment, such method comprises:
a) identifying a particular task for a microbial consortia to modulate,
b) identifying one or more members of the microbial consortia,
c) measuring for one or more of the identified members the translational efficiency (TE) on genes and metabolic pathways,
d) categorizing the identified member into one or more guilds (functional category),
e) identifying one or more microbial niches (i.e., preferred substrates, conditions) for each identified member,
f) analyzing competition interactions between the identified members using distances between microbial niches and guild of members,
g) designing an intervention that modulates the microbial consortia based on competition, guild association and niches, and
h) modulating the microbial consortia.

In certain embodiments, the translational efficiency is determined using genome annotation, transcription and translation information of the identified member. In certain embodiments, the modulation of microbial consortia comprises one or more of the following steps:
a) selectively increasing one or more desired member by providing conditions preferable to the desired member, and/or
b) selectively decreasing one or more undesired members by providing one or more competitor to the desired member or hostile to the desired member or providing conditions preferable to a competitor to the desired member or hostile to the desired member or a combination of both.

In certain embodiments, the modulation improves performance of the microbial consortia to perform the particular task. In certain embodiments, the preferred conditions are based on the niche information and competition is determined by association to guilds.

The present disclosure also provides a method for predicting the effect of perturbations on one or more members of a microbial consortia. In certain embodiments, such method comprises:
a) identifying one or more members of the microbial consortia,
b) measuring for one or more of the identified members the translational efficiency (TE) on genes and metabolic pathways, wherein the translational efficiency is determined using genome annotation, transcription and translation information of the identified member,
c) categorizing the identified member into one or more guilds (functional category),
d) identifying one or more microbial niches (i.e., preferred substrates, conditions) for each identified member,
e) analyzing competition interactions between the identified members using distances between microbial niches and guild of members,
f) predicting the effect of an intervention that modulates the microbial consortia based on competition, guild association and niches, and
g) predicting the effect of an intervention that modulates a composition of the microbial consortia, wherein predictions are based on the niche information and competition is determined by association to guilds.

In certain embodiments, the perturbations comprises changes of an organism's growth (e.g., its absolute or relative abundance, its size, or its growth rate and yield), metabolic activity (e.g., respiration or chemical transformation, antibiotic production, quorum sensing), chemical composition, physical properties (e.g., cell surface properties, surface charge, surface structure, speed of movement, frequency and kind of motion, such as tumbling, gliding, oscillating, production of extracellular matrices), or behavior changes (e.g., association to other organism through physical contact or chemical exchanges).

The present disclosure further provides a method for designing a microbial consortia for a particular task, wherein the members of a microbial consortia are selected from the method disclosed herein. In certain embodiments, a method for selecting one or more members of a microbial consortia for a particular task comprises:
a) identifying one or more members of the microbial consortia,
b) measuring for one or more of the identified members the translational efficiency (TE) on genes and metabolic pathways, wherein the translational efficiency is determined using genome annotation, transcription and translation information of the identified member,
c) categorizing the identified member into one or more guilds (functional category),
d) identifying one or more microbial niches (i.e., preferred substrates, conditions) for each identified member,
e) analyzing competition interactions between the identified members using distances between microbial niches and guild of members,
f) identifying one or more particular task to be performed by one or more members of the microbial consortia, and
g) selecting one or more members of the microbial consortia based on the task to be performed, wherein preferred tasks are based on niche, guild, and competition.

In certain embodiments, the method for selecting one or more members of a microbial consortia for a particular task, wherein the microbial consortia are accompanied by conditions defined based on niche information.

As used herein, a microbial consortia type refers to one or more bacteria (e.g., mycoplasma, coccus, bacillus, rickettsia, spirillum), fungi (e.g., filamentous fungi, yeast), nematodes, protozoans, archaea, algae, dinoflagellates, viruses (e.g., bacteriophages), viroids and/or a combination thereof. In one embodiment, the one or more microorganism strains is one or more bacteria (e.g., mycoplasma, coccus, bacillus, rickettsia, spirillum), fungi (e.g., filamentous fungi, yeast), nematodes, protozoans, archaea, algae, dinoflagellates, viruses (e.g., bacteriophages), viroids and/or a combination thereof. In another embodiment, the one or more microorganism strains is one or more fungal species or fungal subspecies. In another embodiment, the one or more microorganism strains is one or more bacterial species or bacterial subspecies.

As used herein, the microbial consortia originates from animal, human, plant, soil (e.g., bulk soil or rhizosphere), air, saltwater, freshwater, wastewater sludge, built environment, sediment, oil, an agricultural product, an industrial product or process (e.g. fermentation process), a microbial sample, or an extreme environment. In certain embodiments, the animal or human sample is a blood, tissue, tooth, perspiration, fingernail, skin, hair, feces, urine, semen, mucus, saliva, gastrointestinal tract, rumen, muscle, brain, tissue, or organ sample. In certain embodiments, the plant sample is a root, stem, leaf, flower, fruit, seed, xylem, phloem, or juice sample.

As used herein, a member of a microbial consortia comprises one taxonomic unit (e.g., strain, species, genus, family, order, class, phylum, kingdom) present in a microbial consortium as defined above. In certain embodiments, a member of a microbial consortia comprises one single living unit (e.g., one single bacterium, fungus, protozoan, archaea, algae, dinoflagellate, virus, viroid).

As used herein, a composition comprises of any number of different microorganisms (e.g., strain, species, genus, family, order, class, phylum, kingdom) present in a microbial consortium or consortia as defined above. In certain embodiments, a composition comprises one single living unit (e.g., one single bacterium, fungus, protozoan, archaea, algae, dinoflagellate, virus, viroid). In certain embodiments, composition comprises different actives, such as transcriptional, translational, enzymatic, as defined above.

As used herein, the genome annotation comprises nucleic acid sequencing, measuring the number of unique genomic DNA markers and/or a metagenomic approach. In certain embodiments, measuring the number of first unique markers in the sample comprises measuring the number of unique RNA markers. In certain embodiments, measuring the number of unique first markers in the sample comprises measuring the number of unique protein markers. In certain embodiments, measuring the number of unique first markers in the sample comprises measuring the number of unique metabolite markers. In certain embodiments, measuring the number of unique metabolite markers in the sample comprises measuring the number of unique carbohydrate markers, unique lipid markers or a combination thereof.

As used herein, transcriptomics is measuring the level of expression of one or many RNA molecules (e.g., miRNA, tRNA, rRNA, and/or mRNA) in the sample for expression analysis. In certain embodiments, the gene expression analysis comprises a sequencing reaction. In certain embodiments, the RNA expression analysis comprises a quantitative polymerase chain reaction (qPCR), metatranscriptome sequencing, and/or transcriptome sequencing.

As used herein, translation is determining RNA binding to ribosomes by metaribosome profiling, or ribosome profiling. In certain embodiments, measuring translation comprises determining the number of unique proteins, measured by mass spectrometry analysis.

As used herein, the translation efficiency (TE) comprises transcription (e.g., miRNA, tRNA, rRNA, and/or mRNA), and/or translation (e.g., metaribosome profiling, ribosome profiling, or proteomics), and/or a ratio of translation to transcription. In certain embodiments, TE comprises ribosome binding site (RBS) affinity with ribosome (e.g., RBS sequence optimization level).

As used herein, a niche comprises the condition or set of conditions that permit a member or a set of members to exist or to thrive within a microbial consortia or biotope (Hutchinson-Macfayden, 1957), as defined by having high TE level in genome annotated processes (e.g., unique DNA, RNA, protein, or metabolite marker) associated with the said condition or set of condition. In certain embodiments, a niche comprises one substrate or set of substrates (e.g., metabolites, carbohydrates, lipids, proteins, nutrients) for which a member or a set of members have a high TE on processes relative to import, metabolism or processing of the said substrate. In certain embodiments, a niche comprises biochemical, chemical, biophysical, physical or geological conditions for which a member or a set of members have a high TE on genome annotated processes relative to import, metabolism, processing, resistance to the said condition. In certain embodiments, a niche comprises a biological condition (e.g., presence, absence of abundance of an organism or set of organisms) modifying the nutrient, biochemical, biophysical or biogeological conditions associated with high TE on genome annotated processes relative to import, metabolism, processing or resistance to the modified condition.

As used herein, a guild comprises a functional or ecological unit of member or members within microbial consortia characterized based on similarities and distances calculated using a network and/or clustering analysis method to measure distances between each member's TE profiles. In certain embodiments, the clustering analysis comprises network analysis, cluster analysis, linkage analysis, partitioning analysis, variance analysis, or a combination thereof. In another embodiment, distances between members are calculated from the clustering analysis, normalized, Z-scored or a combination thereof. In certain embodiments, the clustering analysis integrates TE measured on annotated genomes (e.g., unique DNA, RNA, protein, or metabolite marker). In certain embodiments, guilds are molded by adaptation to the same class of resources. In certain embodiments, guilds are molded by competition between its members.

As used herein, a competition comprises negative interactions between members using similar resources, where resources are e.g., organic compounds or inorganic compounds and salt, unnatural amino acids, gaseous chemicals, using similar physical properties, e.g., light, temperature, pH, osmolarity, physical space, or using similar biological entities, e.g., living organisms or dead organisms, for maintaining or advancing their life, lifetime, reproduction, growth, and sustainability. In certain embodiments, competition involves the production of antimicrobials, e.g., chemical, biochemical or biophysical compounds to reduce fitness of other members.

As used herein, a predicting effect of perturbation comprises changes of an organism's growth (e.g., its absolute or relative abundance, its size, or its growth rate and yield), metabolic activity (e.g., respiration or chemical transformation, antibiotic production, quorum sensing), chemical composition, physical properties (e.g., cell surface properties, surface charge, surface structure, speed of movement, frequency and kind of motion, such as tumbling, gliding, oscillating, production of extracellular matrices), or behavior changes (e.g., association to other organism through physical contact or chemical exchanges).

As used herein, interventions comprise addition of entire organisms (dead or live) or parts of organisms (e.g., DNA, RNA, proteins or peptides, lipids, cell membranes), physical removal of entire organisms e.g., removal through binding to antibodies, filtration by size exclusion or binding to a matrix, biological removal of entire organisms, e.g., through antibiotics, antimicrobial peptides, predators (e.g., amoeba), or viral infection. In certain embodiments, the interventions comprise changes (i.e., addition, removal or variations in concentrations) of chemical properties (e.g., organic compounds or inorganic compounds and salt, unnatural amino acids, gaseous chemicals). In certain embodiments, the interventions comprise changes (i.e., addition, removal or variations in concentrations) of physical properties, e.g., light, temperature, pH, osmolarity, physical space and environment.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the drawings and supplemental drawings, tables, data and description attached as Appendix I, which is incorporated by reference by its entity. The components in the drawings and supplemental drawings are not necessarily to scale, emphasis instead being placed upon clearly illustrating the principles of the present disclosure. Moreover, in the drawings, like reference numerals designate corresponding parts throughout the several views.

FIGS. 1a-1d show the guild-based microbiome classification of a 16-member microbial community based on translational efficiency (TE). FIG. 1a. Conceptual overview of TE as a readout of functional prioritization for each microbe in a 16-members SynCom of microbes isolated from the switchgrass rhizosphere, where each member has a limited amount of resources (ribosomes) to allocate for protein translation. TE is computed as the metaRibo-Seq/metaRNA-Seq ratio, i.e., the ratio between translated mRNA and total mRNA detected at one given instant in the sample. FIGS. 1b and 1c. TE on 275 KEGG metabolic pathways (n=4 replicates) allowed to classified the 16 SynCom members into 6 different guilds, in which microbes share similar metabolic pathways prioritizations; FIG. 1b. PCA cluster plot and FIG. 1c. dendrogram; FIG. 1d. phylogenetic tree based on 16 S rRNA sequences shows substantial differences with the TE-based guilds dendrogram (FIG. 1c), indicating that guilds are not based solely on phylogeny.

FIGS. 2a-2e show prediction of competition interactions in a microbial community. FIGS. 2a and 2b. a multi-omics profiling of a 16-strains soil SynCom was performed; FIG. 2b. relative abundances of the 16 members of the SynCom at the metagenomics, metatranscriptomics and metaRibo-Seq levels, color key d) applies; FIG. 2c. metaRS and metaT profiles were used to compute TE and classify the members into metabolic guilds as described in FIGS. 1b-1c; FIG. 2d. a competition score was computed to predict competitive interactions against each SynCom member based on the proximity of its guild with each other member's. High competition scores (plus signs) indicate SynCom members (in columns) that are likely to compete with the targeted member (in row). Asterisks indicate competition against targeted members that have been tested experimentally as shown in FIG. 2e; FIG. 2e. five individual members were experimentally dropped out from the SynCom prior to incubation (asterisks), and relative abundances of all remaining members were compared to the non-modified SynCom. Graphics show linear regression and 99% confidence interval (CI, in gray) of square-transformed relative abundances (RPKM) in the SynCom (x-axis) vs. single dropout SynCom (y-axis). Organisms above (arrows) the 99% CI were considered significantly increased in response to the dropout, thus showing that they were competing with the dropped out member in the SynCom. In all the tested conditions, significantly increased members were accurately predicted to compete with the dropped out member (high competition scores against the targeted member in d, rowwise).

FIG. 3 shows interventions of addition of microorganisms based on competition prediction. Burkholderia-Rhizobium and Mucilaginibacter-Chitinophaga were predicted to be strong guild competitors in the SynCom (FIG. 2d); linear regression and 99% confidence interval (CI) of square-transformed relative abundances in control (x axis) vs. modified community (y axis). Organisms above/below the 99% CI were considered significantly increased/decreased in the modified community; top row, left to right: Burkholderia-Rhizobium competition testings: Burkholderia dropout increased Rhizobium; Burkholderia (20×) enrichment decreased Rhizobium; Rhizobium (20×) enrichment decreased Burkholderia; Burkholderia-Rhizobium double dropout had very minor effect on the composition of the rest of the community; bottom row, left to right: Burkholderia-Rhizobium competition testings: Mucilaginibacter dropout increased Chitinophaga; Mucilaginibacter (100×) enrichment decreased Chitinophaga; Chitinophaga (100×) enrichment decreased Mucilaginibacter; Mucilaginibacter-Chitinophaga double dropout did not have any effect on the composition of the rest of the community.

FIG. 4 shows TE for genes coding metabolite import proteins in a community for the identification of niches. Darker shades indicate higher TE values.

FIG. 5 shows the effects of metabolite addition on community composition. Linear regression and 99% CI of metagenomics relative abundances (RPKM, log scaled) in 0.1×R2A control (x axis, n=2) versus 0.1×R2A+substrate (y axis, n=2). Organisms above or below the 99% CI are considered as significantly increased or decreased upon addition of substrate. Upwards arrows indicate organisms with a high TE for import protein for the tested substrate (primary targets, see FIG. 4) that were successfully increased. Downwards arrows indicate competitors that were successfully decreased (secondary targets, see FIG. 2d). Addition of (from left to right, top to bottom): fructose, galactose, maltose+maltodextrin, ribose, trehalose, xylose, putrescine, spermidine, glutamate, glutathione, sulfate+thiosulfate. Data from the most effective of the tested concentrations are shown.

FIG. 6 shows that MiND and guilds accurately predict the effect of combined pre- and probiotic intervention in soil. Linear regression and 99% CI of metagenomics relative abundances (RPKM, log scaled) in soil+SynCom probiotic (x axis) versus soil+SynCom probiotic+prebiotic (y axis). Organisms above or below the 99% CI are considered as significantly increased or decreased upon addition of substrate. Upwards arrows indicate successfully increased primary targets, downward arrows indicate successfully decreased secondary targets. Addition of fructose (left) and ribose (right) induced an increase in Burkholderia's relative abundance (high TE for fructose and ribose import proteins, see FIG. 4), and a decrease of its competitors Rhizobium and Variovorax (high competition scores against Burkholderia, see FIG. 2d).

FIG. 7 shows the application of the guild classification method to a human microbiome sample. Dendrogram of TE measured on metabolic pathways in a natural gut microbiome sample from a healthy human.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

The present disclosure provides a new method integrating transcriptional and translational regulation measurements, revealing how each microbe allocates its resources for optimal proteome efficiency. Protein translation is the most expensive process in a cell, so microbes closely regulate their resource allocation by prioritizing essential functions through differential translational efficiency (TE) (AI-Bassam et al. 2018). Thus, direct measurement of TE in a microbial community sample would shine light on the metabolic role of each member of the community and allow to better understand interactions with other members.

In certain embodiments, the present disclosure provides a method for a design and intervention of microbial consortia comprising the following steps:
a) identifying a particular task for a microbial consortia to modulate,
b) identifying one or more members of the microbial consortia,
c) measuring one or more of the identified members the translational efficiency (TE) on genes and metabolic pathways,
d) categorizing the identified member into one or more guilds (functional category),
e) identifying one or more microbial niches (i.e., preferred substrates, conditions) for each identified member,
f) analyzing competition interactions between the identified members using distances between microbial niches and guild of members,
g) designing an intervention that modulates the microbial consortia based on competition, guild association and niches, and
h) modulating the microbial consortia.

In certain embodiments, the translational efficiency is determined using genome annotation, transcription and translation information of the identified member. In certain embodiments, the modulation of microbial consortia comprises one or more of the following steps:
a) selectively increasing one or more desired member by providing conditions preferable to the desired member, and/or
b) selectively decreasing one or more undesired members by providing one or more competitor to the desired member or hostile to the desired member or providing conditions preferable to a competitor to the desired member or hostile to the desired member or a combination of both.

In certain embodiments, the modulation improves performance of the microbial consortia to perform the particular task. In certain embodiments, the preferred conditions are based on the niche information and competition is determined by association to guilds.

A method for predicting the effect of perturbations on one or more members of a microbial consortia and a method for designing microbial consortia for a particular task are also provided herein. In certain embodiments, the perturbations comprises changes of an organism's growth (e.g., its absolute or relative abundance, its size, or its growth rate and yield), metabolic activity (e.g., respiration or chemical transformation, antibiotic production, quorum sensing), chemical composition, physical properties (e.g., cell surface properties, surface charge, surface structure, speed of movement, frequency and kind of motion, such as tumbling, gliding, oscillating, production of extracellular matrices), or behavior changes (e.g., association to other organism through physical contact or chemical exchanges). In certain embodiments, the microbial consortia are accompanied by conditions defined based on niche information.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a catalyst,” “a metal,” or “a substrate,” includes, but are not limited to, mixtures or combinations of two or more such catalysts, metals, or substrates, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’, where ‘x’ and ‘y’ are numerical values, includes “about ‘x’ to about ‘y’.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Unless otherwise specified, temperatures referred to herein are based on atmospheric pressure (i.e., one atmosphere).

In certain embodiments of the present disclosure, metatranscriptomics and metatranslatomics analysis were performed to directly measure TE in situ, in a 16-member synthetic community (SynCom) compiled from rhizosphere isolates grown in a complex culture medium. It allowed to perform a guild-based microbiome classification, grouping microbes according to the metabolic pathways they prioritize, independently of their taxonomic relationships. It was shown that guilds predicted competition between members of the same guild with 100% sensitivity and 74% specificity (77% accuracy) in the SynCom. Further, gene-level analysis of TE allowed to predict each microbe's substrate preferences, i.e., their niche in the community. Such Microbial Niche Determination (MiND) successfully predicted which particular microbes would benefit from substrates supplementation with 54% sensitivity and 83% specificity (78% accuracy) in the SynCom. As microbes adapt their translational regulation to community settings, such predictions could not be achieved using axenic culture approaches (i.e., phenotypic microarray, growth curves), or partially functional measurements (i.e., metagenomics, metatranscriptomics).

Further, combining TE-based MiND and guilds predictions allowed to selectively manipulate the SynCom, by increasing or decreasing abundance of targeted members either by providing preferred substrates or by giving an advantage to their competitors. Most importantly, the method disclosed herein is scalable to more complex, natural samples. In certain embodiments, MiND was applied to native soil and human fecal samples and its applicability to predict changes and manipulate microorganisms in complex microbiomes were demonstrated.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Methods & Materials

### Isolates

A 16-member microbial SynCom was established from the rhizosphere of switchgrass (Panicum virgatum) from agricultural crops, consisting of one strain each of Arthrobacter, Bosea, Bradyrhizobium, Brevibacillus, Burkholderia, Chitinophaga, Lysobacter, Marmoricola, Methylobacterium, Mucilaginibacter, Mycobacterium, Niastella, Paenibacillus, Rhizobium, Rhodococcus and Variovorax (Coker et al. 2022). These isolates were obtained from the rhizosphere and soil surrounding a single switchgrass plant grown in marginal soils described elsewhere (Ceja-Navarro et al. 2021). Isolates and details on their isolation are available from the Leibniz Institute German Collection of Microorganisms and Cell Cultures GmbH (DSMZ) under accession numbers DSM 113524 (Arthrobacter OAP107), DSM 113628 (Bosea OAE506), DSM 113701 (Bradyrhizobium OAE829), DSM 113525 (Brevibacillus OAP136), DSM 113627 (Burkholderia OAS925), DSM 113563 (Chitinophaga OAE865), DSM 113522 (Lysobacter OAE881), DSM 114042 (Marmoricola OAE513), DSM 113562 (Mucilaginibacter OAE612), DSM 113602 (Methylobacterium OAE515), DSM 113539 (Mycobacterium OAE908), DSM 113593 (Niastella OAS944), DSM 113526 (Paenibacillus OAE614), DSM 113517 (Rhizobium OAE497), DSM 113518 (Rhodococcus OAS809), DSM 113622 (Variovorax OAS795).

### Isolates Growth Conditions

Precultures of individual isolates were made in 5 mL of liquid 1×R2A medium (Teknova, cat #R0005) in aerobic conditions at 30° C. for 7 days, without shaking. Note, one isolate (Bradyrhizobium OAE829) was grown in 0.1×R2A due to poor growth in 1×R2A, as described previously (Coker et al. 2022).

### SynCom Assembly and Growth Conditions

Optical density readings at 600 nm (OD₆₀₀), from isolates pre-cultures were taken with a Molecular Devices SpectraMax M3 Multi-Mode Microplate Reader (VWR, cat #89429-536). Precultures were diluted to a starting OD₆₀₀ of 0.02 in 5 mL 0.1×R2A. The SynCom was assembled in large volumes to minimize pipetting error and maximize reproducibility. Briefly, 1 mL of each normalized culture (OD₆₀₀=0.02) was diluted in a final volume of 250 mL 0.1×R2A and spread into 20 mL aliquots in Falcon tubes. Falcon tubes containing the SynCom inoculum were then incubated at 30° C. for 7 days, in aerobic conditions, in four biological replicates. After 7 days of culture, the SynCom samples were harvested by centrifugation and pellets were immediately treated for multi-omics analysis as detailed below (DNA-, RNA- and metaRibo-Seq).

### Targeted Interventions in the SynCom

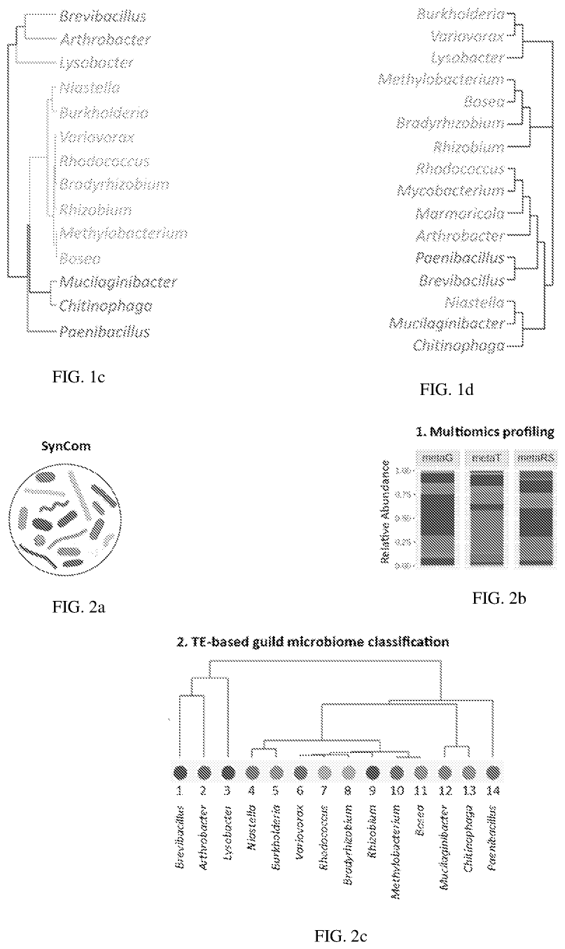

For targeted modifications experiments carried out in the SynCom, the SynCom was assembled and cultured as described above, with modifications. Specific isolates were omitted from the SynCom assembly for the dropout experiments, as described in FIGS. 2d and 2e. Alternatively, concentrated stocks of either Burkholderia, Chitinophaga, Mucilaginibacter or Rhizobium were added as probiotics to the SynCom. Metabolites from concentrated, filter-sterilized stocks of the desired substrates were added to the medium for the prebiotics experiments. Modified SynCom were then incubated at 30° C. for 7 days, in aerobic conditions, in two biological replicates. After 7 days of culture, the modified SynCom samples were harvested by centrifugation and pellets were stored at −80° C. prior to metagenomics analysis.

### Natural Soil Growth Conditions

A soil associated with switchgrass lowland reference genome clone (Missaoui, Boerma, and Bouton 2005) that was sampled from a field located in Texas, USA (28.3325, −98.1175) was used. Fifty gram (50 g) of soil was added to 250 mL of 0.1×R2A culture medium or 0.1×R2A+SynCom inoculum prepared as described above, and this volume was spread in 14 mL culture tubes (5 mL in each tube). 50-500 μL of concentrated, filter-sterilized stocks of the desired substrates and/or 20 μL of the SynCom isolate pure cultures diluted at an OD₆₀₀ of 0.02 (i.e. approximately 8.10⁵ CFU/mL) were then added. Soil samples were then grown at 30° C. for 7 days, in aerobic conditions, in two biological replicates (three replicates for reference soil sample). After 7 days of culture, the soil samples were harvested by centrifugation and pellets were stored at −80° C. prior to metagenomics analysis.

### Metatranslatomics (MetaRibo-Seq) Sample Preparation

MetaRibo-Seq sample preparation was performed as detailed in the protocol provided below. This protocol shares similarities with the recently published MetaRibo-Seq protocol from Fremin et al. 2021, with modifications (Fremin, Sberro, and Bhatt 2020). Briefly, bacterial lysis was performed in a solution containing Chloramphenicol to stop protein elongation. Monosome recovery was performed following MNase treatment, using RNeasy Mini spin size-exclusion columns (Qiagen) and RNA Clean & Concentrator-5 kit (Zymo). rRNA removal was performed using the QIAseq FastSelect-5S/16S/23S kit (Qiagen). MetaRibo-Seq libraries were prepared using the NEBNext Small RNA Library Prep set for Illumina, with modifications. Amplification was followed in real time using SYBR-Green and stopped when reaching a plateau. PCR products were purified using Select-a-size DNA Clear & Concentrator kit (Zymo). Leftover lysate prior to MNase treatment was saved and stored at −80° C. for metagenomics and metatranscriptomics analysis.

### Metagenomics (DNA-Seq) and Metatranscriptomics (RNA-Seq) Sample Preparation

DNA and RNA from SynCom samples were extracted from leftover lysates from metaRibo-Seq sample preparation, stored in Trizol at −80 C. DNA from soil samples was extracted using ZymoBIOMICS DNA miniprep kit (Zymo). DNA-Seq libraries were prepared using Nextera XT library preparation kit with 700 pg DNA input per sample and 6:30 min tagmentation at 55° C. and barcoded using Nextera XT indexes (Illumina). RNA was extracted using RNeasy mini kit (Qiagen), and rRNA was removed using QIAseq FastSelect-5S/16S/23S kit (Qiagen). RNA-Seq libraries were prepared using KAPA RNA HyperPrep kit (Roche) and barcoded using TruSeq indexes (Illumina). Amplification was followed in real time using SYBR-Green and stopped when reaching a plateau.

### Sequencing

The quality and average size of the libraries was controlled using a 4200 TapeStation System (Agilent). Libraries concentrations were quantified using Qubit dsDNA HS Assay kit and QuBit 2.0 Fluorometer (Invitrogen). Libraries were pooled by -omic and experiment and sequenced on a Illumina NovaSeq, PE100 platform. Minimum sequencing depth was 10 million reads for metagenomics samples, 50 million reads for metatranscriptomics samples, and 100 million reads for metatranslatomics samples.

### Reference Genomes

Genomics data from individual cultures of the 16 SynCom members were used to assemble genomes using FLASh2 merging and SPAdes, and quality controlled using CheckM. Genomes were annotated at the gene level using PROKKA version 1.14.5 (Seemann 2014), and KEGG pathway annotation was performed using BlastKOALA version 2.2 (Kanehisa, Sato, and Morishima 2016). A custom SynCom metagenome database was built from the 16 isolates' genomes using bowtie2 version 2.3.2 (Langmead and Salzberg 2012).

### Data Processing

Adapter sequences were removed from multi-omics sequencing data using TrimGalore (Cutadapt version 1.18), and quality controlled using FastQC version 0.11.9 (Andrews et al. 2010). Trimmed reads were aligned to a custom SynCom database using bowtie2 version 2.3.2. Gene count tables were obtained using Woltka version 0.1.1 (Zhu et al. 2022). Multi-omics gene counts were normalized to reads per kilobase per million (RPKM).

### Statistics

Feature count tables were imported and analyzed using R version 3.6.3. HCPC analysis was performed using the FactomineR package (Lê, Josse, and Husson 2008). Graphical representations were performed using the ggplot2, gplots, cowplot and factoextra packages (Wickham 2016; Irnawati et al. 2020).

### TE Calculation and Guild Clustering

TE for each of the 275 KEGG pathways present amongst the 16 SynCom members was calculated as the ratio between Ribo-Seq and RNA-Seq reads per kilobase per million (RPKM). Hierarchical clustering on the principal components (HCPC) (Le, Josse, and Husson 2008) of TE data allowed to group community members according to the metabolic pathways they prioritize, i.e. their guild.

TE was computed as:

[symbol]

TE [symbol] _(i,j)=Ribo-Seq [symbol] _(i,j)/ [symbol] RNA-Seq [symbol] _(i,j);

Where Ribo-Seq_{i,j} and RNA-Seq_{i,j} are Ribo-Seq and RNA-Seq RPKM for each feature i in each bacteria j. TE_{i,j} was calculated at two different levels: i) considering features as KEGG pathway and ii) considering features as genes. KEGG-pathway analysis was performed on all Ribo-Seq_{i,j} and RNA-Seq_{i,j}>10 RPKM.

### Competition Score

For each pair of bacteria_{i,j}, we defined a competition score_{i,j}, referring to the likelihood of bacteria j increasing upon removal of bacteria i, as a function of the distance between i and j in the guild clustering as follows:

[symbol]

Score [symbol] _(i,j)=−( [symbol] dist [symbol] _(i,j)−“μ”_i)/“σ”_i;

Where dist_{i,j} is the euclidean distance between bacteria i and j in the guild clustering, μᵢ and σᵢ are the average and the standard deviation of the population distances to bacteria i. Score_{i,j} was then adjusted to zero for bacteria having a relative abundance <0.5% after 7 days of growth in the SynCom.

Sensitivity and specificity of the prediction of community outcomes upon modifications of the community composition were calculated by considering a Score_{i,j}>0 as “likely” and Score_{i,j}<0 as “unlikely” for bacteria j to increase upon removal of bacteria i from the SynCom.

### Phenotypic Microarray Assays for Axenic Culturing of Isolates

All 16 SynCom member isolates were grown axenically on 285 different substrates using Biolog Phenotypic Microarray (PM) plates (PM 1, 2A, 3B) following company instructions (Biolog). Briefly, isolates were streaked on 1×R2A agar plates (1.5% w/v) (Bradyrhizobium was streaked on 0.1×R2A plates), colonies picked and resuspended in inoculation fluid IF0a GN/GP (Biolog Part #72268) up to an OD600 of 0.07 and inoculated into the microarrays in triplicate. Microarrays were stored at 30 C incubator without shaking, with lids coated with an aqueous solution of 20% ethanol and 0.01% Triton X-100 (Sigma, cat #X100-100 ML) to prevent condensation (Coker et al. 2022). Absorbance readings were taken at 0 hr and 144 hr time points. Growth in PM2A wells were indicated by blank subtracted OD600 increases greater than 0.02, which was the minimum absorbance reached by all isolates in 0.1×R2A at the start of their exponential growth phase. PM1 and PM3B assays were performed in the same way but supplemented with 1×RedoxDyeMix G (Biolog Part #74227) (for gram negative isolates) or RedoxDyeMix H (Part #74228) (for gram positive isolates) with growth in wells indicated by increases greater than 0.02 in blank subtracted OD590 readings. Axenic growth capabilities on these substrates were used to train individual GEM models for all 16 isolates.

### GEMs Simulations

Genome-scale metabolic models (GEMs) of 16 SynCom members were simulated on in silico media that includes the uptake fluxes of metabolites using Flux Balance Analysis (FBA). The predicted growth rates of SynCom members were recorded for comparison purposes. To predict the effect of media supplemented with different substrates, we incorporated the uptake flux of each substrate at a time and simulated the model for each change in the media. The models were analyzed using COBRApy software package (version 0.17.1) with IBM CPLEX solver (version 22.1.0) (IBM) in Python (version 3.7.11).

### Example 2

### Guild-Based Microbiome Classification

Protein translation is the most expensive process in a cell, and bacteria use translational regulation to accurately allocate finite resources and prioritize functions essential for their adaptation (AI-Bassam et al. 2018; Le Scornet and Redder 2019; Fris and Murphy 2016). Ribosome profiling (Ribo-Seq), i.e. translatomics, allows the direct measurement of protein translation in vivo in real time (Latif et al. 2015). It was shown that translational efficiency (TE), calculated by analyzing the number of ribosomes on a given transcript as the ratio between translated mRNA over total mRNA (Ribo-Seq/RNA-Seq), can be used as a direct readout of functional prioritization in axenic bacterial cultures (AI-Bassam et al. 2018).

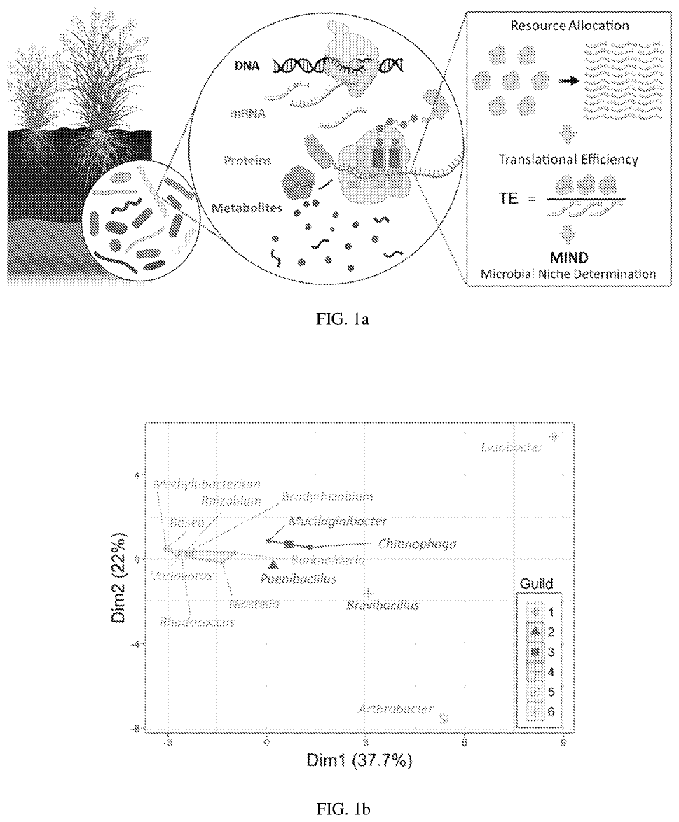

Here, metagenomics, metatranscriptomics and metatranslatomics (also called metaribosome profiling or metaRibo-Seq) (Fremin, Sberro, and Bhatt 2020) were applied to simultaneously measure TE in multiple organisms in a 16-member microbial community from rhizosphere isolates grown in complex medium (see methods, FIG. 1a). Multi-omics experiments showed excellent reproducibility between biological replicates and highlighted strong differences between metagenomics, -transcriptomics, and -translatomics data. TE on metabolic pathways allowed to classify microbes into functional guilds, revealing each microbe's metabolic role in the community (see methods, FIGS. 1b and 1c). The 16-member SynCom was divided into 6 guilds, defined by specific metabolic functions (i.e., pathway prioritization) that separate them from the rest of the community (FIGS. 1b and 1c). For example, Lysobacter (guild 6) has a significantly higher TE for denitrification and dissimilatory nitrate reduction compared to the other guilds, while Chitinophaga and Mucilaginibacter (guild 3) have a high TE for assimilatory sulfate reduction, thiosulfate oxidation and multiple antimicrobial resistance pathways.

Metabolic pathway prioritization was different when bacteria were grown axenically or in the SynCom, showing the importance of performing functional analysis in community settings directly, rather than in isolated cultures. On another hand, TE-based metabolic guilds were substantially different from phylogenetic clustering, showing that they define functional categories that are independent of taxonomic relationships (FIG. 1d). A similar analysis based on genome content, metatranscriptomics or metaRibo-Seq data did not resemble TE-based clusterings. These data hint at current limitations of 16 S rRNA and genome-based approaches that infer function and activity from phylogeny or genomes.

### Example 3

### Guilds Predicts Bacterial Competition

As early as 1967, Root defined ecological guilds—observed outside of the microbial world at this time—as functional categories molded by adaptation to the same class of resources, but also by competition between its members. To proof such within-guild competition also applied to microbes, individual members were dropped out from the SynCom and the effect on relative abundance of the remaining 15 members was evaluated. FIG. 2b shows the relative abundance of each of the 16 SynCom members at the metagenomics, metatranscriptomics, and metaRibo-Seq level (FIGS. 2a and 2b). A guild-based competition score was computed based on the guild clustering distance matrix, that similar guilds would predict competitive interactions (see methods, FIGS. 2c and 2d). In 5/5 tested conditions, a single microbe dropout benefitted at least one of its closest competitors from the same guild, which relative abundance increased significantly more than the rest of the community (FIGS. 2d and 2e). For example, when Mucilaginibacter was removed, it was observed that two of its closest neighbors in the guilds clustering (Chitinophaga and Burkholderia) increased in abundance (FIG. 2e, bottom right). Similarly, removal of Burkholderia resulted in an increase of its close competitor Rhizobium (FIG. 2e, middle right). Comparable results were obtained for all single strain removal experiments, i.e., removal of Arthrobacter, Bradyrhizobium, Burkholderia, Lysobacter and Mucilaginibacter (FIG. 2e). Elevated metabolic activity was also observed based on metatranscriptomics and metatranslatomics, for microbes with increased abundance. Overall, guild-based competition scores allow to predict competitive interactions within the microbial community with excellent sensitivity (100%) and specificity (74%) (77% accuracy).

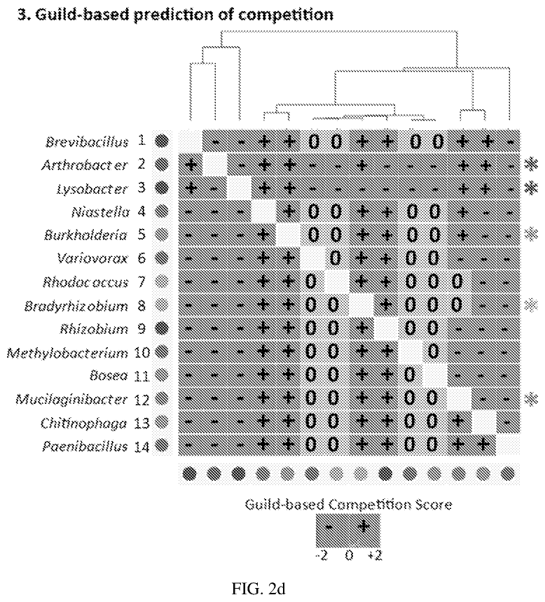

To further validate the observed competition interactions, an in-depth analysis was conducted on two of the strong competition pairs observed in the dropout experiments (i.e., Chitinophaga-Mucilaginibacter and Burkholderia-Rhizobium, FIG. 2e and FIG. 3). It was observed that adding a more concentrated inoculum of each of these strains to the SynCom (similar to a probiotic intervention), resulted in a very specific decrease in relative abundance of their main competitor (FIG. 3). On the other hand, removing both members of these competition pairs had very little to no effect on the abundance of microbes from the other guilds (FIG. 3), suggesting that competition is constrained into each guild. spot-on-lawn assays were then performed to screen for antimicrobial compounds produced by these competition pairs. In-line with guild-based competition predictions, Chitinophaga was observed to specifically inhibit the growth of Mucilaginibacter, while it failed to inhibit the growth of any other SynCom member. This suggests the production of a narrow-spectrum antimicrobial by Chitinophaga targeted against its guild competitor Mucilaginibacter. In contrast, Burkholderia and Rhizobium did not inhibit each other's or any other member's growth, indicating that the mechanism of competition within this guild is likely not mediated by constitutively expressed antimicrobial compounds.

Overall, members of the same guild specifically compete between each other. Removal of a competitor allows another member of this guild to fill that niche, while addition of a competitor would have the opposite effect. It was also observed production of microbial compounds by one SynCom member, specifically targeted against its closest competitor. This confirms that TE-based metabolic guilds accurately predict competition interactions in a microbial community. Of note, a similar analysis based on metatranscriptomics data alone did not efficiently predict competition (37.5% sensitivity and 76% specificity only).

### Example 4

### Microbial Niche Determination (MiND)

Next, TE was used to identify substrate preferences, i.e., metabolites that would specifically increase the abundance of targeted members of the SynCom, akin to a prebiotic. High TE for genes coding for import proteins would indicate prioritized metabolism for the corresponding substrates, thus defining a microbe's niche.

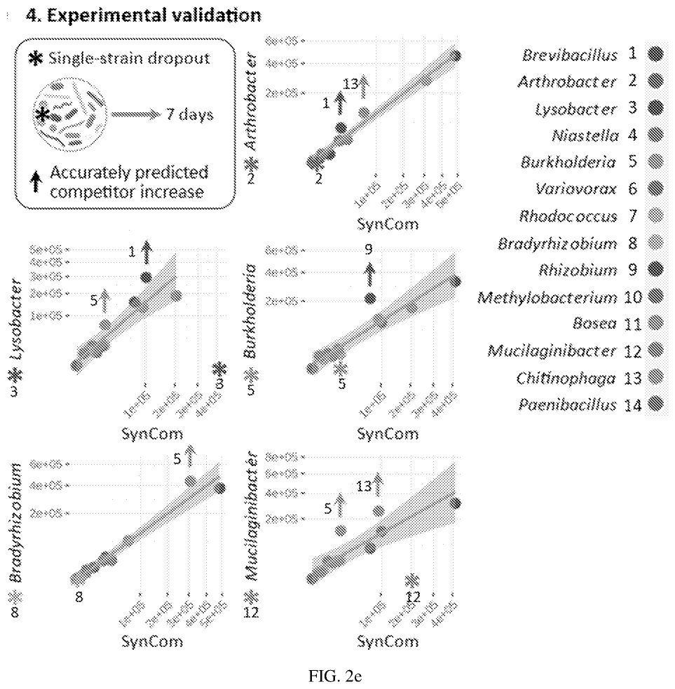

A total of 88 genes coding for import proteins were detected in the SynCom at the metagenomics level, of which 40 (45%) were expressed and translated based on metatranscriptomics and metaRibo-Seq data. MiND was performed by calculating TE for each of these 40 import protein genes in each SynCom member, thus defining their substrate preferences, i.e., their niche (FIG. 4). Based on this analysis, 14 metabolites were selected to be tested as prebiotics in the SynCom.

Phenotypic microarray (Biolog) confirmed the ability of each SynCom member to utilize MiND-predicted preferred substrates in isolation, with 88% of high TE measured in the SynCom being confirmed in isolation by Biolog. Ability to utilize a substrate in axenic culture, however, did not necessarily translate into a high priority for this substrate's intake in the SynCom, thus only 33% of substrate use abilities detected by Biolog in axenic conditions translated into a high TE in the SynCom. Similar results were observed by comparing axenic growth curves of each SynCom member in 0.1×R2A+selected substrate with the MiND predictions. This shows that while bacteria have the ability to utilize a range of substrates in axenic cultures, they only prioritize a fraction of them once put in more complex community settings.

### Example 5

### MiND Predicts Effects of Substrate Addition

Culture medium was supplemented with metabolites identified in the MiND analysis, in order to specifically benefit microbes that prioritize the import of these substrates (primary targets). Furthermore, metabolite-induced increase of targeted bacteria would result in a concomitant decrease of this bacteria's nearest guild competitors (secondary targets).

A total of 14 different compounds were tested in three different concentrations, including six sugars (fructose, galactose, maltose/maltodextrin, ribose, trehalose, xylose), three amino acids (cystine, glutamate, methionine), two diamines (putrescine, spermidine), one vitamin (cobalamin), one peptide (glutathione), and one inorganic compound (sulfate/thiosulfate). Addition of cobalamin, cystine or methionine did not induce any significant change in relative abundance in the community, likely because these substrates were already present in excess in the non-modified culture medium and were thus discarded from the analysis.

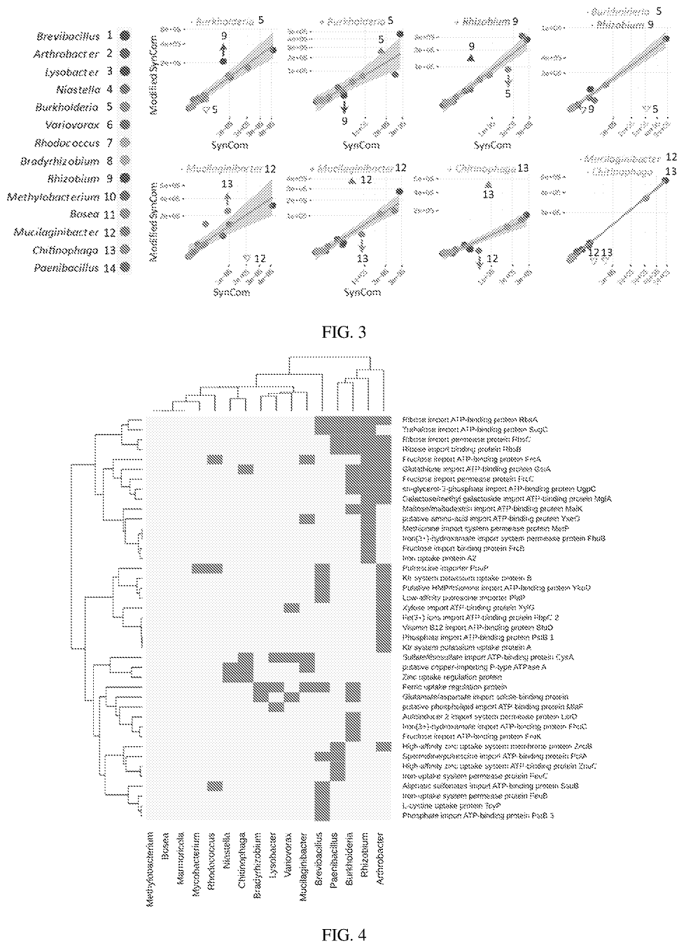

Bacteria with high TE for a given metabolite's import protein were specifically increased in relative abundance after metabolite addition (FIG. 4 and FIG. 5). At the same time, when primary target microbes were increased in relative abundance, competitors from the same guild decreased. For example, addition of ribose induced a specific increase of primary targets Paenibacillus and Burkholderia (primary targets), which showed the highest TE for ribose importers in the SynCom, while inducing a specific decrease of Burkholderia's competitors Variovorax, Rhizobium, and Bradyrhizobium (secondary targets) (FIGS. 1b and 1c; FIG. 2d; and FIG. 5). Similarly, addition of glutathione specifically increased primary targets Burkholderia and Chitinophaga, both having a high TE for the glutathione import protein GsiA, while decreasing their competitors Mucilaginibacter, and Bradyrhizobium (FIGS. 1 b and 1c; FIG. 2d; and FIG. 5), and addition of putrescine specifically increased Paenibacillus and Rhodococcus, which both had a high TE for putrescine import proteins PuuP and PotA, while reducing competitors Rhizobium, Bradyrhizobium, and Mucilaginibacter (FIGS. 1b and 1c; FIG. 2d; and FIG. 5). Overall, MiND predicted the specific increased relative abundance of the prebiotic's primary target(s) for 9/11 tested prebiotics, with 54% sensitivity and 83% specificity (78% accuracy). In all of these 9 cases, the successful increase of primary targets also resulted in a decrease of at least one of their competitors. Guild classification predicted such competition-based decrease of secondary targets with 93% sensitivity and 65% specificity (70% accuracy).

In conclusion, combining MiND and guild classification accurately predicts substrate preferences and competition in a 16-members SynCom, and thus can be used to design and predict the outcome of targeted interventions in a microbial community.

### Example 6

### Genome-Scale Metabolic Models Improve Accuracy of MiND Predictions

MiND and guild analyses predict which SynCom members are likely to increase in relative abundance upon substrate addition, and which competitions influence the outcome of prebiotic intervention. However, if two competitors both prioritize the import of a specific substrate, the outcome of this competition could not be predicted, as observed for the competition pair Burkholderia-Rhizobium, both prioritizing ribose import (FIG. 4). To further assess competition outcome on a given substrate, genome-scale metabolic models (GEMs) of the 16 SynCom members were deployed, to simulate growth in silico, with and without addition of the above selected substrates (see methods). GEMs predicted that both Burkholderia and Rhizobium would have a higher growth rate upon addition of ribose, which was confirmed by Biolog phenotypic microarray and growth curves in axenic cultures. Yet, the difference in growth rate is higher for Burkholderia than for Rhizobium, thus it was accurately predicted that Burkholderia would outcompete Rhizobium in the SynCom (FIG. 3). GEMs accurately predicted the effect of ribose addition on other primary targets (high TE for ribose import proteins) Paenibacillus (increased), Arthobacter and Brevibacillus (not increased) (FIG. 3). Overall, GEMs refining allowed to increase specificity and accuracy of TE-based MiND predictions of primary target increase upon addition of sugars.

### Example 7

### MiND Predicts Interventions Outcome in Soil

After benchmarking MiND for the 16-member SynCom, it was evaluated if principles observed from the SynCom study can be extrapolated to the soil environment, one of the most complex microbiomes. MiND predicted interactions and prebiotic intervention outcomes were tested in non-sterile natural soil samples. In analogy to results from the SynCom study (FIGS. 2d and 2e), it was observed that bacteria with high TE for import proteins were specifically increased (primary targets, FIG. 4) and these bacteria's closest competitors were decreased as defined by MiND (secondary targets, FIG. 2d).

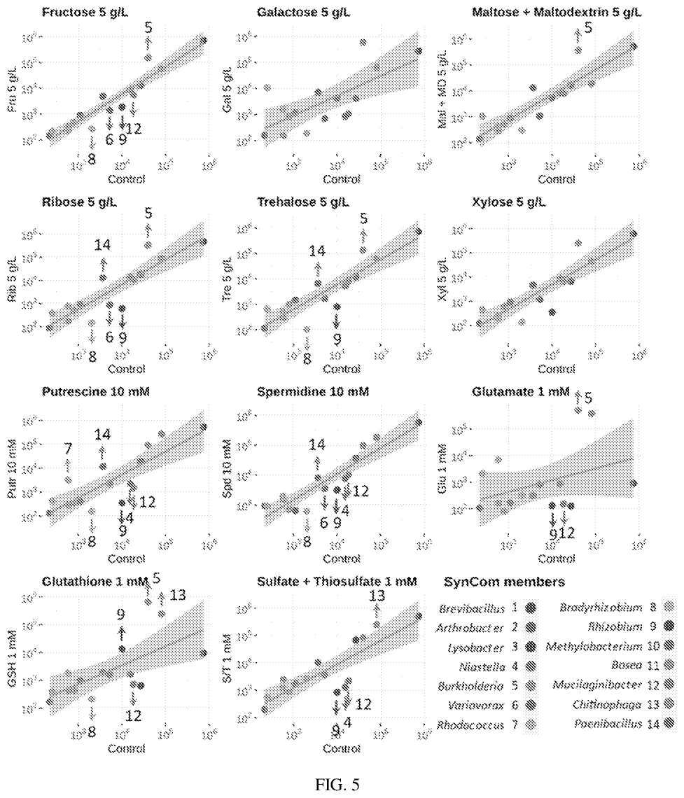

Soil samples from a switchgrass crop similar to the site from which the SynCom strains were originally isolated, were incubated in 33 different conditions: 0.1×R2A alone (soil control), with and without metabolite additions (prebiotic), and with and without individual SynCom members (probiotic, 8.10⁵ CFU/mL), or the whole 16-member SynCom (probiotic consortium, 8.10⁵ CFU/mL of each member) (see methods and FIG. 6). Changes in microbial community composition were analyzed by shotgun metagenomics.

The SynCom members resulted in on average 1% of all metagenomic reads, showing that these SynCom members were detectable in the soil samples, but did not dominate the rhizosphere microbiome. This proportion was higher in the soil+probiotic consortium samples, while decreasing overtime (SynCom members making on average 6.8, 4.6, 2.6 and 1.6% of total reads after 2, 4, 7 and 14 days of incubation), showing the transitional nature of probiotic intervention outcome. Data showed good reproducibility between replicates.

As an example, the relative abundance of Burkholderia in the natural soil samples was sought to increase, through either i) single probiotic intervention (Burkholderia); ii) combined pre- and single probiotic (fructose+Burkholderia); iii) combined prebiotic and probiotic consortium (fructose+SynCom); or iv) prebiotic-only treatment (fructose). The results show that i) probiotic treatment alone induced a non-significant increase of Burkholderia (fold change=1.3, FIG. 6); ii) combined pre- and probiotic treatment with fructose+Burkholderia and iii) fructose+SynCom also successfully increased Burkholderia (fold change=3.9 and 5.3, respectively) while reducing competitors (FIG. 6); iv) prebiotic-only treatment with fructose, also induced a significant increase in Burkholderia's relative abundance (fold change=3.9), together with a decrease of its closest competitors Rhizobium and Variovorax (FIG. 6).

Similarly, it was aimed to increase Chitinophaga and predict its closest competitor, Mucilaginibacter, to decrease. Probiotic treatment alone successfully increased Chitinophaga (fold change=12.3), while inducing a non-significant decrease of Mucilaginibacter (FIG. 6). Chitinophaga+glutathione combined treatment induced a large and specific increase of Chitinophaga (fold change=37.6), while significantly decreasing Mucilaginibacter (FIG. 6), which was also observed in SynCom+glutathione. Of note, glutathione treatment alone did not increase Chitinophaga in soil, probably because of its very low initial abundance (FIG. 6). Glutathione treatment did not decrease Mucilaginibacter abundance either, confirming that Mucilaginibacter's decrease observed in the Chitinophaga+glutathione condition is a result of competition against Chitinophaga, rather than a direct effect of glutathione on Mucilaginibacter(FIG. 6). These results were in good agreement to observations made in the SynCom alone (FIG. 5), suggesting that niches are stable under the same conditions, independent of the community size.

In 5 out of 7 conditions tested, and despite the very low amount of probiotic added (OD₆₀₀=0.00008, i.e., about 8×10⁵ CFU/mL), probiotic intervention successfully increased the primary target, without dominating the rest of the community. In 10 out of 10 tested conditions, prebiotic+single probiotic treatment resulted in a successful increase of the primary target, together with a predicted decrease of secondary targets in 8 out of these 10 cases. Prebiotic+probiotic consortium (SynCom) treatment also increased the primary target in 6/7 tested conditions, with a successful decrease of secondary targets in all these 6 cases. Thus, prebiotic supplementations allowed to modulate the effect of the probiotic consortium, by increasing either Burkholderia through the addition of SynCom+fructose, or Chitinophaga through the addition of SynCom+glutathione, for example. Finally, prebiotic treatment alone successfully increased the predicted primary targets in 3 out of 7 conditions tested and decreased the secondary targets in 3 out of these 3 cases.

Combined prebiotic and probiotic, or prebiotic treatments alone often outperformed results from probiotic treatment alone. For example, Paenibacillus probiotic treatment alone did not significantly increase its abundance (fold change=1.5). However, combined prebiotic and probiotic treatment with Paenibacillus's+preferred substrates substantially increased Paenibacillus and caused a 28.8-fold change for Paenibacillus+ribose and 14.7 fold-change for Paenibacillus+trehalose. Prebiotic treatment with ribose and trehalose alone resulted in a smaller but significant increase of Paenibacillus (fold change=3.5 and 5.5, respectively).

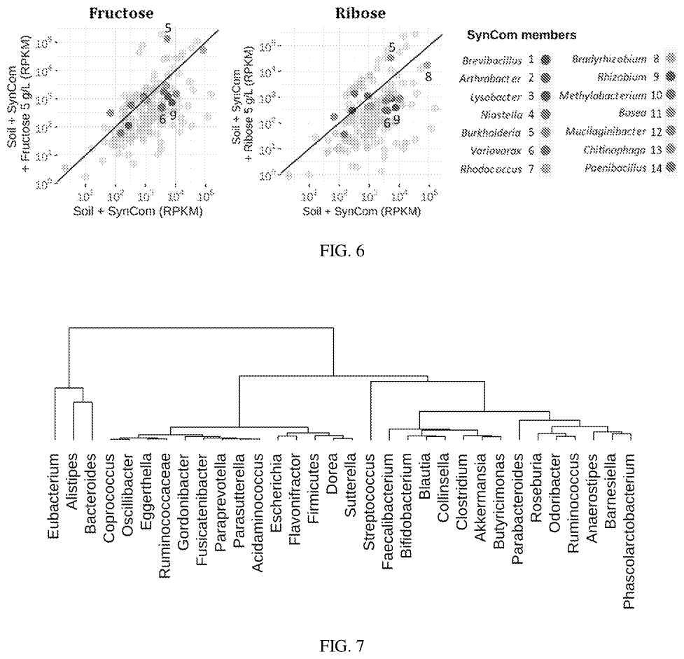

These results also indicate that the strong competition interactions identified in the SynCom only experiments (i.e., Burkholderia—Rhizobium, Chitinophaga—Mucilaginibacter) are maintained in soil conditions. As such, it was repeatedly observed that a successful increase of primary target Burkholderia results in a decrease of secondary targets Rhizobium and Variovorax, across multiple experiments both in SynCom (FIGS. 2d and 2e; FIG. 3 and FIG. 5) and in native soil (FIG. 6). Although Burkholderia outcompeted Rhizobium on most tested substrates, the opposite effect, i.e., Rhizobium outcompeting Burkholderia, was successfully induced using Rhizobium+glutathione combined treatment in soil. To demonstrate broad applicability of the method, MiND was applied to human fecal samples and guilds in these sample revealed, indicating targets for interventions (FIG. 7).

Overall, the results presented herein reveal the potential of MiND to identify and predict bacterial competition, and to design and predict the effect of targeted interventions in complex communities.

### REFERENCES

AI-Bassam, Mahmoud M., Ji-Nu Kim, Livia S. Zaramela, Benjamin P. Kellman, Cristal Zuniga, Jacob M. Wozniak, David J. Gonzalez, and Karsten Zengler. 2018. “Optimization of Carbon and Energy Utilization through Differential Translational Efficiency.” Nature Communications 9 (1): 4474.
Ceja-Navarro, Javier A., Yuan Wang, Daliang Ning, Abelardo Arellano, Leila Ramanculova, Mengting Maggie Yuan, Alyssa Byer, et al. 2021. “Protist Diversity and Community Complexity in the Rhizosphere of Switchgrass Are Dynamic as Plants Develop.” Microbiome 9 (1): 96.
Coker, Joanna, Kateryna Zhalnina, Clarisse Marotz, Deepan Thiruppathy, Megan Tjuanta, Gavin D'Elia, Rodas Hailu, et al. 2022. “A Reproducible and Tunable Synthetic Soil Microbial Community Provides New Insights into Microbial Ecology.” mSystems 7 (6): e0095122.
Fremin, Brayon J., Hila Sberro, and Ami S. Bhatt. 2020. “MetaRibo-Seq Measures Translation in Microbiomes.” Nature Communications 11 (1): 3268.
Fris, Megan E., and Erin R. Murphy. 2016. “Riboregulators: Fine-Tuning Virulence in Shigella.” Frontiers in Cellular and Infection Microbiology 6 (January): 2.
Irnawati, Irnawati, Florentinus Dika Octa Riswanto, Sugeng Riyanto, Sudibyo Martono, and Abdul Rohman. 2020. “The Use of Software Packages of R Factoextra and FactoMineR and Their Application in Principal Component Analysis for Authentication of Oils.” Indonesian Journal of Chemometrics and Pharmaceutical Analysis, July, 1.
Kanehisa, Minoru, Yoko Sato, and Kanae Morishima. 2016. “BlastKOALA and GhostKOALA: KEGG Tools for Functional Characterization of Genome and Metagenome Sequences.” Journal of Molecular Biology 428 (4): 726-31.
Langmead, Ben, and Steven L. Salzberg. 2012. “Fast Gapped-Read Alignment with Bowtie 2.” Nature Methods 9 (4): 357-59.
Latif, Haythem, Richard Szubin, Justin Tan, Elizabeth Brunk, Anna Lechner, Karsten Zengler, and Bernhard O. Palsson. 2015. “A Streamlined Ribosome Profiling Protocol for the Characterization of Microorganisms.” BioTechniques 58 (6): 329-32.
Le Scornet, Alexandre, and Peter Redder. 2019. “Post-Transcriptional Control of Virulence Gene Expression in Staphylococcus Aureus.” Biochimica et Biophysica Acta, Gene Regulatory Mechanisms 1862 (7): 734-41.
Lê, Sébastien, Julie Josse, and Frangois Husson. 2008. “FactoMineR: An R Package for Multivariate Analysis.” Journal of Statistical Software 25 (March): 1-18.
Missaoui, A. M., H. R. Boerma, and J. H. Bouton. 2005. “Genetic Variation and Heritability of Phosphorus Uptake in Alamo Switchgrass Grown in High Phosphorus Soils.” Field Crops Research 93 (2-3): 186-98.
Seemann, Torsten. 2014. “Prokka: Rapid Prokaryotic Genome Annotation.” Bioinformatics (Oxford, England) 30 (14): 2068-69.
Wickham, Hadley. 2016. “Getting Started with Ggplot2.” In Use R!, 11-31. Cham: Springer International Publishing.
Zhu, Qiyun, Shi Huang, Antonio Gonzalez, Imran McGrath, Daniel McDonald, Niina Haiminen, George Armstrong, et al. 2022. “Phylogeny-Aware Analysis of Metagenome Community Ecology Based on Matched Reference Genomes While Bypassing Taxonomy.” MSystems 7 (2): e0016722.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1.-41. (canceled)

42. A method for a design and intervention of a microbial consortia, comprising:
a) identifying a particular task for the microbial consortia to modulate,
b) identifying one or more members of the microbial consortia,
c) measuring for one or more of the identified members a translational efficiency (TE) on genes and metabolic pathways, wherein the translational efficiency (TE) comprises genome annotation, transcription, translation, or a ratio of translation to transcription;
d) categorizing the identified member into one or more guilds,
e) identifying one or more microbial niches for each identified member,
f) analyzing competition interactions between the identified members using distances between microbial niches and guild of members,
g) designing an intervention that modulates the microbial consortia based on competition, guild association and niches, and
h) modulating the microbial consortia.

43. The method of claim 42, wherein the niche comprises a condition or a set of conditions that permit a member or a set of members to exist or to thrive within a microbial consortia.

44. The method of claim 42, wherein the guild comprises a functional or ecological unit of member or members within the microbial consortia characterized based on similarities and distances calculated using a network and/or clustering analysis method to measure distances between each member's TE profiles.

45. The method of claim 42, wherein the microbial consortia is modulated by one or more of the following steps:
a) selectively increasing one or more desired member by providing conditions preferable to the desired member, and
b) selectively decreasing one or more undesired members by providing one or more competitor to the desired member or hostile to the desired member or providing preferred conditions to a competitor to the desired member or hostile to the desired member, or a combination thereof, wherein the preferred conditions are based on the niche information and competition is determined by association to guilds.

46. The method of claim 45, wherein the preferred conditions are based on the niche information and competition is determined by association to guilds.

47. The method of claim 45, wherein the competition comprises negative interactions between members using similar resources, similar physical properties, and/or similar biological entities.

48. The method of claim 42, wherein modulation improves performance of the microbial consortia to perform the particular task.

49. A method for predicting an effect of perturbations on one or more members of a microbial consortia, comprising:
a) identifying one or more members of the microbial consortia,
b) measuring for one or more of the identified members a translational efficiency (TE) on genes and metabolic pathways, wherein the translational efficiency (TE) comprises genome annotation, transcription, translation, or a ratio of translation to transcription,
c) categorizing the identified member into one or more guilds,
d) identifying one or more microbial niches for each identified member,
e) analyzing competition interactions between the identified members using distances between microbial niches and guild of members,
f) predicting the effect of an intervention that modulates the microbial consortia based on competition, guild association and niches, and
g) predicting the effect of an intervention that modulates a composition of the microbial consortia.

50. The method of claim 49, wherein perturbations comprise one or more changes of an organism's growth, metabolic activity, chemical composition, physical properties, or behavior changes.

51. The method of claim 49, wherein the niche comprises a condition or a set of conditions that permit a member or a set of members to exist or to thrive within a microbial consortia.

52. The method of claim 49, wherein the guild comprises a functional or ecological unit of member or members within the microbial consortia characterized based on similarities and distances calculated using a network and/or clustering analysis method to measure distances between each member's TE profiles.

53. The method of claim 49, wherein the competition comprises negative interactions between members using similar resources, similar physical properties, and/or similar biological entities.

54. A method for selecting one or more members of a microbial consortia for a particular task, said method comprising:
a) identifying one or more members of the microbial consortia,
b) measuring for one or more of the identified members a translational efficiency (TE) on genes and metabolic pathways, wherein the translational efficiency (TE) comprises genome annotation, transcription, translation, or a ratio of translation to transcription,
c) categorizing the identified member into one or more guilds,
d) identifying one or more microbial niches for each identified member,
e) analyzing competition interactions between the identified members using distances between microbial niches and guild of members,
f) identifying one or more particular task to be performed by one or more members of the microbial consortia, and
h) selecting one or more members of the microbial consortia based on the task to be performed.

55. The method of any one of claims 42, 49, or 54, wherein the microbial consortia comprises one or more bacteria, nematodes, protozoans, archaea, algae, dinoflagellates, viruses, viroids, or a combination thereof.

56. The method of claim 55, wherein the microbial consortia originates from animal, human, plant, soil, air, saltwater, freshwater, wastewater sludge, built environment, sediment, oil, an agricultural product, an industrial product or process, a microbial sample, or an extreme environment.

57. The method of claim 54, wherein the niche comprises a condition or a set of conditions that permit a member or a set of members to exist or to thrive within a microbial consortia.

58. The method of claim 54, wherein the guild comprises a functional or ecological unit of member or members within the microbial consortia characterized based on similarities and distances calculated using a network and/or clustering analysis method to measure distances between each member's TE profiles.

59. The method of claim 54, wherein the competition comprises negative interactions between members using similar resources, similar physical properties, and/or similar biological entities.
